(19) United States
(12) Patent Application Publication
(10) Pub. No.: US 2021/0145580 A1
(43) Pub. Date: May 20, 2021

(54) POST-IMPLANTATION TENSIONING IN CARDIAC IMPLANTS

(71) Applicant: 4Tech Inc., Waltham, MA (US)

(72) Inventor: Patrick Griffin, Galway (IE)

(73) Assignee: 4Tech Inc., Waltham, MA (US)

(21) Appl. No.: 16/960,205

(22) PCT Filed: Aug. 7, 2018
(86) PCT No.: PCT/US2018/045523

§ 371 Date: Jul. 6, 2020

(51) Int. Cl. A61F 2/24

(52) U.S. Cl. CPC A61F 2/2442; A61F 2220/0008; A61F 2220/0075

(57) ABSTRACT

A heart-treatment system (100) includes a first tissue anchor (30) and a second tissue anchor (140), which includes a stent (158), which includes (a) a plurality of struts (160) arranged as a tubular stent body (161) and (b) a locking frame (184). One or more tethers (32) couple together the first and the second tissue anchors (30, 140). A longitudinal portion (180) of the one or more tethers (32) passes through one or more openings (182) of the locking frame (184) so as to form a tether loop (186). The system (100) is arranged such that enlargement of the tether loop (186) by pulling on the tether loop (186) applies tension between the first and the second tissue anchors (30, 140). Other embodiments are also described.

### FIELD OF THE APPLICATION

The present invention relates generally to minimally-invasive valve repair, and more specifically to minimally-invasive methods for repairing the tricuspid valve.

### BACKGROUND OF THE APPLICATION

Functional tricuspid regurgitation (FTR) is governed by several pathophysiologic abnormalities such as tricuspid valve annular dilatation, annular shape, pulmonary hypertension, left or right ventricle dysfunction, right ventricle geometry, and leaflet tethering. Treatment options for FTR are primarily surgical.

U.S. Pat. No. 8,475,525 to Maisano et al. describes a method that includes implanting at least a first tissue-engaging element in a first portion of tissue in a vicinity of a heart valve of a patient, implanting at least a second tissue-engaging element in a portion of a blood vessel that is in contact with an atrium of a heart of the patient, and drawing at least a first leaflet of the valve toward at least a second leaflet of the valve by adjusting a distance between the portion of the blood vessel and the first portion of tissue in the vicinity of the heart valve of the patient. In one configuration, a proximal end portion of a longitudinal member is shaped so as to define one or more engaging elements (e.g., hooks or barbs), which are coupleable with the struts of a stent member in order to maintain the tension applied to a longitudinal member for remodeling the tricuspid valve.

### SUMMARY OF THE APPLICATION

Some applications of the present invention provide a system for treating a heart of a patient, the system comprising a first tissue anchor configured to be implanted in cardiac tissue of the patient, and a second tissue anchor configured to be implanted in the patient. One or more tethers couple together the first and the second tissue anchors. A longitudinal portion of the one or more tethers passes through one or more openings of a locking frame so as to form a tether loop. Enlargement of the tether loop by pulling on the tether loop applies tension between the first and the second tissue anchors. For some applications, the second tissue anchor comprises a stent, which comprises a plurality of struts arranged as a tubular stent body. For some of these applications, the stent further comprises the locking frame.

Some applications of the present invention provide a method of treating a heart of a patient, such as to reduce tricuspid valve regurgitation. The method includes implanting a first tissue anchor in cardiac tissue of the patient and a second tissue anchor in the patient, such that the first and the second tissue anchors are coupled together by one or more tethers. Thereafter, tension is applied between the first and the second tissue anchors using at least a longitudinal portion of the one or more tethers. Typically, the one or more tethers are slack before the tension is applied. For some applications, the tension is applied after allowing at least 24 hours for tissue growth on the first tissue anchor to strengthen anchoring of the first tissue anchor in the cardiac tissue, while for other applications, the tension is applied during the same surgical session in which the first and the second tissue anchors are implanted.

There is therefore provided, in accordance with an application of the present invention, a system for treating a heart of a patient, including:

a first tissue anchor, which is configured to be implanted in cardiac tissue of the patient;

a second tissue anchor, which (a) is configured to be implanted in the patient, and (b) includes a stent, which includes (i) a plurality of struts arranged as a tubular stent body and (ii) a locking frame; and

one or more tethers that couple together the first and the second tissue anchors,

wherein a longitudinal portion of the one or more tethers passes through one or more openings of the locking frame so as to form a tether loop, and

wherein the system is arranged such that when the first tissue anchor is implanted in the cardiac tissue and the second tissue anchor is implanted in the patient, enlargement of the tether loop by pulling on the tether loop applies tension between the first and the second tissue anchors.

For some applications, the locking frame is integral with the tubular stent body.

For some applications, the longitudinal portion of the one or more tethers pass through exactly one opening of the locking frame so as to form the tether loop.

For some applications, the first tissue anchor is configured to penetrate the cardiac tissue.

For any of the applications described above, the longitudinal portion of the one or more tethers that forms the tether loop may be an end longitudinal portion of the one or more tethers. For some applications, the end longitudinal portion is fixed to the locking frame. For some applications, the end longitudinal portion is fixed to the tubular stent body.

For any of the applications described above, the locking frame may be shaped so as define a base and a deflectable tab, and the longitudinal portion of the one or more tethers passes between the base and the deflectable tab. For some applications, the deflectable tab is shaped so as to define serrations.

For any of the applications described above, the stent may include a flop-prevention sleeve disposed inside the tubular stent body, and the tether loop is disposed partially within the flop-prevention sleeve.

There is further provided, in accordance with an application of the present invention, a system for treating a heart of a patient, including:

a first tissue anchor configured to be implanted in cardiac tissue of the patient;

a second tissue anchor configured to be implanted in the patient;

one or more tethers that couple together the first and the second tissue anchors; and

a locking frame, wherein a longitudinal portion of the one or more tethers passes through one or more openings of the locking frame so as to form a tether loop,

wherein the system is arranged such that when the first tissue anchor is implanted in the cardiac tissue and the second tissue anchor is implanted in the patient, enlargement of the tether loop by pulling on the tether loop applies tension between the first and the second tissue anchors.

For some applications, the second tissue anchor includes a stent, which includes a plurality of struts arranged as a tubular stent body. For some applications, the stent further includes the locking frame. For some applications, the locking frame is integral with the tubular stent body. For some applications, the longitudinal portion of the one or more tethers passes through exactly one opening of the locking frame so as to form the tether loop. For some applications, the longitudinal portion of the one or more tethers that forms the tether loop is an end longitudinal portion of the one or more tethers. For some applications, the end longitudinal portion is fixed to the locking frame. For some applications, the end longitudinal portion is fixed to the tubular stent body.

For some applications, the locking frame is shaped so as define a base and a deflectable tab, and the longitudinal portion of the one or more tethers passes between the base and the deflectable tab. For some applications, the deflectable tab is shaped so as to define serrations.

For some applications, the stent includes a flop-prevention sleeve disposed inside the tubular stent body, and the tether loop is disposed partially within the flop-prevention sleeve.

For some applications, the first tissue anchor is configured to penetrate the cardiac tissue.

For some applications, the longitudinal portion of the one or more tethers passes through two openings of the locking frame so as to form the tether loop.

There is still further provided, in accordance with an application of the present invention, a method of treating a heart of a patient, including:

implanting a first tissue anchor in cardiac tissue of the patient and a second tissue anchor in the patient, such that the first and the second tissue anchors are coupled together by one or more tethers and a longitudinal portion of the one or more tethers passes through one or more openings of a locking frame so as to form a tether loop; and

thereafter, applying tension between the first and the second tissue anchors by enlarging the tether loop by pulling on the tether loop.

For some applications, the second tissue anchor includes a stent, which includes a plurality of struts arranged as a tubular stent body, and implanting the second tissue anchor includes implanting the stent in a blood vessel.

For some applications, the blood vessel is selected from the group consisting of: a superior vena cava (SVC), an inferior vena cava (IVC), and a coronary sinus.

For some applications, the stent further includes the locking frame.

For some applications, the locking frame is integral with the tubular stent body.

For some applications, the longitudinal portion of the one or more tethers passes through exactly one opening of the locking frame so as to form the tether loop.

For some applications, the longitudinal portion of the one or more tethers that forms the tether loop is an end longitudinal portion of the one or more tethers.

For some applications, the end longitudinal portion is fixed to the locking frame.

For some applications, the end longitudinal portion is fixed to the tubular stent body.

For some applications, the locking frame is shaped so as define a base and a deflectable tab, and the longitudinal portion of the one or more tethers passes between the base and the deflectable tab.

For some applications, the deflectable tab is shaped so as to define serrations.

For some applications, the stent includes a flop-prevention sleeve disposed inside the tubular stent body, and the tether loop is disposed partially within the flop-prevention sleeve.

For some applications, implanting the first tissue anchor includes penetrating the first tissue anchor into the cardiac tissue.

For some applications, the longitudinal portion of the one or more tethers passes through two openings of the locking frame so as to form the tether loop.

For some applications, the one or more tethers are slack before applying the tension.

For some applications, applying the tension includes applying the tension after allowing at least 24 hours for tissue growth on the first tissue anchor to strengthen anchoring of the first tissue anchor in the cardiac tissue.

For some applications, implanting the first tissue anchor includes implanting the first tissue anchor in the vicinity of the tricuspid valve of the patient.

The present invention will be more fully understood from the following detailed description of embodiments thereof, taken together with the drawings, in which:

### BRIEF DESCRIPTION OF THE DRAWINGS

FIGS. 1A-E are schematic illustrations of a technique of treating a heart of a patient, in accordance with an application of the present invention;

FIGS. 2A-D are schematic illustrations of another technique for treating a heart of a patient, in accordance with an application of the present invention;

FIG. 3 is a schematic illustration of a system for treating a heart of a patient, in accordance with an application of the present invention;

FIGS. 4A-B are schematic illustrations of a technique for treating the heart using the system of FIG. 3, in accordance with an application of the present invention;

FIG. 5 is a schematic illustration of a portion of another system for treating a heart of a patient, in accordance with an application of the present invention; and

FIGS. 6A-B are schematic illustrations of a technique for treating the heart using the system of FIG. 5, in accordance with an application of the present invention.

### DETAILED DESCRIPTION OF APPLICATIONS

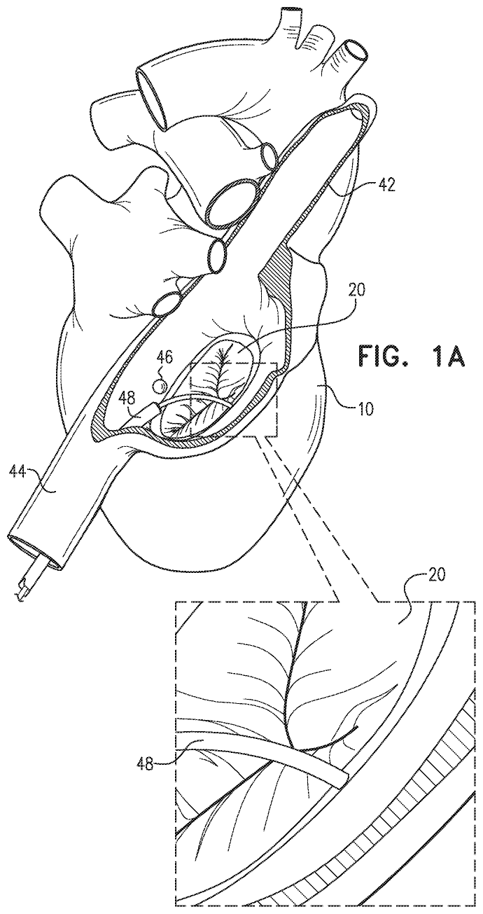

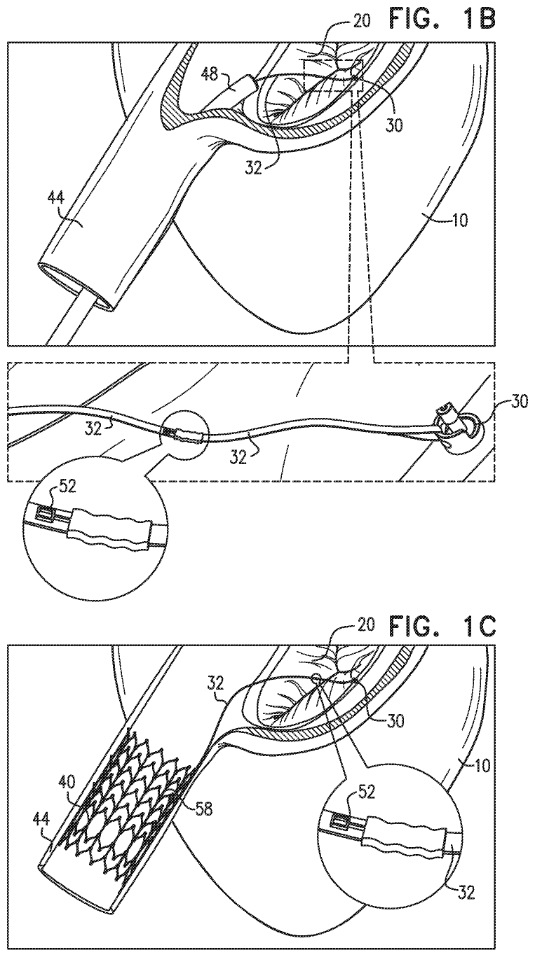

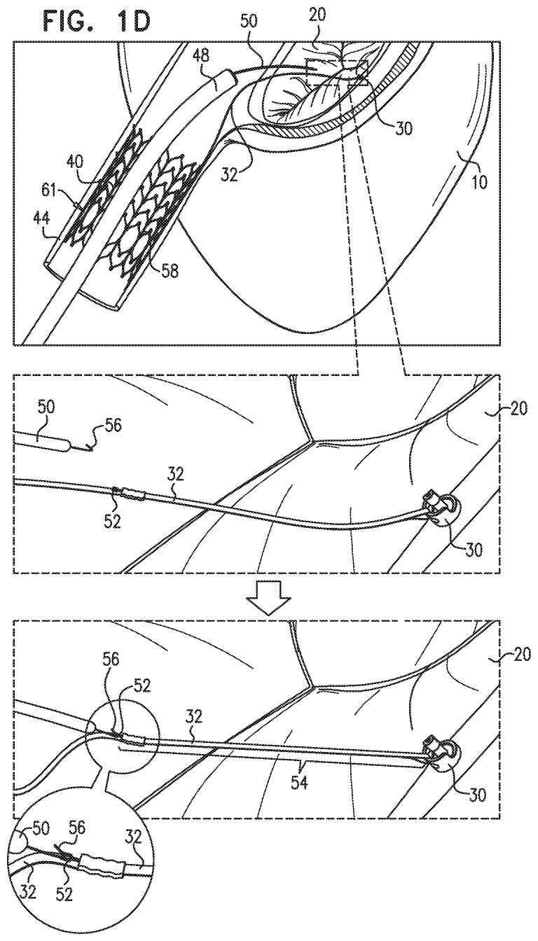

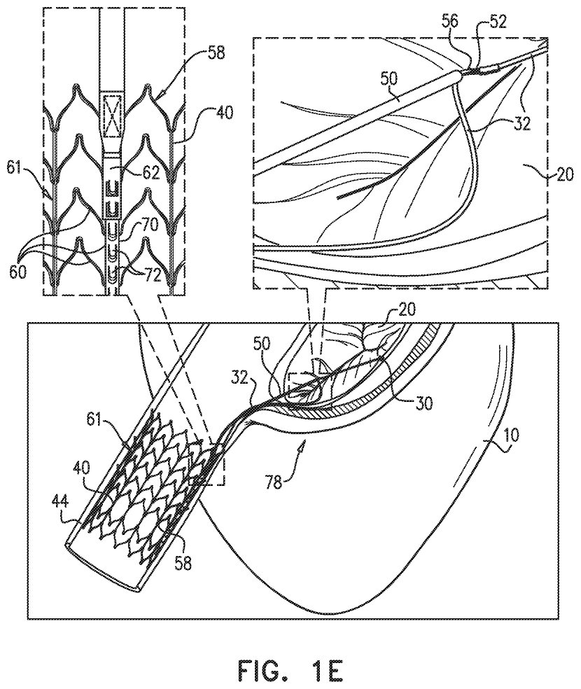

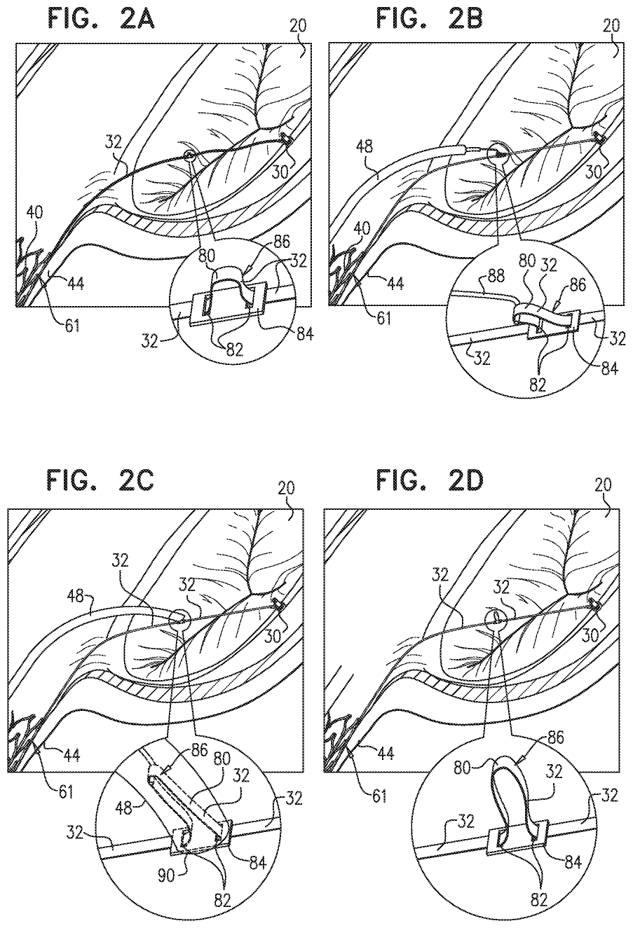

FIGS. 1A-E are schematic illustrations of a technique for treating a heart 10 of a patient, in accordance with an application of the present invention. FIGS. 2A-D, 3, 4A-B, 5, and 6A-B are schematic illustrations of additional techniques for treating heart 10, in accordance with an application of the present invention. For some applications, the techniques of FIGS. 1A-E, 2A-D, 3, 4A-B, 5, and 6A-B are used to treat a tricuspid valve 20, such as by reducing tricuspid valve regurgitation.

As shown in FIGS. 1A-C, 2A, and 4A, during a first stage of an implantation procedure, a first tissue anchor 30 is implanted in cardiac tissue of the patient, and a second tissue anchor 40 is implanted in the patient, either before or after implanting first tissue anchor 30, such that first and second tissue anchors 30 and 40 are coupled together by one or more tethers 32. For example, first tissue anchor 30 may be implanted in the vicinity of tricuspid valve 20 (as shown), e.g., on or near the annulus, and/or second tissue anchor 40 may be implanted in a superior vena cava (SVC) 42, an inferior vena cava (IVC) 44 (as shown), or a coronary sinus 46. Typically, first and second tissue anchors 30 and 40 are implanted in a transcatheter procedure (typically endovascularly, such as percutaneously), via one or more catheters 48, such as described in the applications incorporated hereinbelow by reference. Optionally, the one or more tethers 32 comprise two tethers 32 that are coupled together in situ during the first stage of the implantation procedure, such as using techniques described in one or more of the applications incorporated by reference hereinbelow, e.g., using the techniques described with reference to FIGS. 20-26 of U.S. Pat. No. 9,307,980. Typically, first tissue anchor 30 is configured to penetrate the cardiac tissue. For some applications, first tissue anchor 30 comprises a helical tissue-anchoring element, or one of the anchors described in PCT Publications WO 2016/087934 and/or WO 2016/18939, which are incorporated herein by reference.

For some applications, at this first stage of the implantation procedure, the one or more tethers 32 are slack, i.e., do not apply tension between first and second tissue anchors 30 and 40.

As shown in FIGS. 1D-E, 2B-D, and 4B, thereafter, during a second stage of the implantation procedure, tension is applied between first and second tissue anchors 30 and 40 using at least a longitudinal portion of the one or more tethers 32. Typically, application of the tension remodels tricuspid valve 20, by drawing two or three of the leaflets together to enhance coaptation.

For some applications, the tension is applied after allowing at least 24 hours (e.g., at least one week, such as at least one month) for tissue growth (e.g., fibrous and/or endothelial tissue growth) on first tissue anchor 30 to strengthen anchoring of first tissue anchor 30 in the cardiac tissue. For some applications, the tension is applied within two months after implanting first tissue anchor 30.

For other applications, the implantation procedure is a single surgical session that includes both the first stage (during which first tissue anchor 30 and second tissue anchor 40 are implanted) and the second stage (during which the tension is applied). For example, the tension may be applied within three hours after implanting the first tissue anchor.

For applications in which second tissue anchor 40 comprises a stent 58, such as described below, use of the techniques described herein may allow the use of longer tether(s) 32 than would otherwise be possible. Using longer tether(s) 32 may afford greater flexibility in selecting the axial location for implantation of stent 58 in SVC 42, IVC 44, or coronary sinus 46, because the axial location can be selected without regard to the exact desired tension that will ultimately be applied between the first and the second tissue anchors. In addition, the flexibility of selecting the axial location without regard to the exact desired tension may allow releasing the entire stent from a delivery catheter essentially at once, rather than retaining a portion of the stent radially compressed to allow axial motion of the stent after partial release in order to set the tension. The release of the stent from the delivery catheter essentially at once may allow the use of a shorter stent than would be necessary for staged release from the delivery catheter.

For some applications, such as shown in FIGS. 1A-E, the one or more tethers 32 are one or more first tethers 32, and applying the tension comprises (a) coupling a second tether 50 to a coupling site 52 along the one or more first tethers 32, and (b) applying the tension between the first and the second tissue anchors 30 and 40 using at least a longitudinal portion 54 (labeled in FIG. 1D) of the one or more first tethers 32 and second tether 50. Typically, longitudinal portion 54 extends from coupling site 52 to first tissue anchor 30.

For some applications, coupling second tether 50 to coupling site 52 comprises coupling, to coupling site 52, a coupling element 56 that is attached to second tether 50. For example, coupling element 56 may comprise a hook, as shown in FIGS. 1D-E.

Typically, applying the tension comprises coupling second tether 50 to second tissue anchor 40. For some applications, as shown, second tissue anchor 40 comprises a stent 58 that comprises a plurality of struts 60 arranged as a tubular stent body 61, and coupling second tether 50 to second tissue anchor 40 comprises coupling, to one or more of struts 60, a coupling element 62 that is attached to second tether 50. For some applications, coupling element 62 is shaped so as to define an opening 64, e.g., exactly one opening 64 or a plurality of openings 64, arranged, for example, axially along coupling element 62. Optionally, opening 64 is defined by a loop of coupling element 62. Providing a plurality of openings 64 provides redundancy; in case one of the openings does not catch on stent 58, another of the openings may catch (or both may catch, as shown in the figures). Optionally, coupling element 62 comprises one or more loops that are shaped so as to define the one or more openings 64, respectively.

For some applications, at least one of struts 60 is oriented axially (i.e., along the axis of the stent) as a backbone 70 (which may be thicker, wider, and/or stronger than other struts 60 and/or other axially-oriented struts 60). Coupling element 62 is coupled to backbone 70. In one configuration, backbone 70 is shaped so as to define one or more hooks 72 (e.g., exactly one hook 72, or two or more hooks 72), and coupling element 62 is coupled to one or more of hooks 72. For example, coupling element 62 may be shaped so as to define one or more openings 64, as described above. Providing a plurality of hooks 72 provides redundancy, as discussed above regarding openings 64. Alternatively, for some applications, coupling element 62 comprises one or more hooks, which are hooked onto one or more of struts 60, such as backbone 70.

Reference is made to FIG. 1E. For some applications, a system 78 for treating heart 10 is provided. System 78 comprises first and second tissue anchors 30 and 40, and the one or more first tethers 32 that couple together first and second tissue anchors 30 and 40. System 78 further comprises second tether 50, which is configured to be coupled to coupling site 52 along the one or more first tethers 32, so as to apply tension between first and second tissue anchors 30 and 40 using (a) second tether 50 and (b) longitudinal portion 54 of the one or more first tethers 32. (Longitudinal portion 54 typically extends from coupling site 52 to first tissue anchor 30.) For some applications, system 78 further comprises coupling element 56 that is attached to second tether 50 and is configured to be coupled to coupling site 52 along the one or more first tethers 32. System 78 may alternatively or additionally comprise any of the other elements and/or features described hereinabove with reference to FIGS. 1A-E.

For some applications, such as shown in FIGS. 2A-D, a longitudinal portion 80 of the one or more tethers 32 passes through one or more openings 82 of a locking frame 84 so as to form a tether loop 86. In the configuration shown in FIGS. 2A-D, longitudinal portion 80 is a non-end longitudinal portion 80 of the one or more tethers 32, i.e., longitudinal portion 80 does not extend to any ends of the one or more tethers 32. As shown in FIGS. 2B-D, the tension is applied by enlarging tether loop 86 by pulling on tether loop 86. For example, a hooking element 88 may be introduced, for example, through a catheter 48, and used to temporarily snag tether loop 86 and pull on the tether loop. (The catheter may be introduced through the same vena cava as during the first stage of the implantation procedure, or through the other vena cava.) A distal end 90 of catheter 48 may be held against locking frame 84 to provide a counterforce for pulling on the tether loop. For some applications, openings 82 are configured to provide sufficient friction to prevent tether loop 86 from contracting when ordinary tensions are applied to the one or more tethers 32. For some applications, such as shown in FIGS. 2A-D, longitudinal portion 80 of the one or more tethers 32 passes through two openings 82 of locking frame 84 so as to form tether loop 86.

Reference is made to FIG. 2D. For some applications, a system 94 for treating heart 10 is provided. System 94 comprises first and second tissue anchors 30 and 40, and the one or more tethers 32 that couple together first and second tissue anchors 30 and 40. Longitudinal portion 80 of the one or more tethers 32 passes through the one or more openings 82 of locking frame 84 so as to form tether loop 86. System 94 is arranged such that when first tissue anchor 30 is implanted in the cardiac tissue and second tissue anchor 40 is implanted in the patient, enlargement of tether loop 86 by pulling on tether loop 86 applies tension between first and the second tissue anchors 30 and 40 (using at least a longitudinal portion of the one or more tethers 32). System 94 may alternatively or additionally comprise any of the other elements and/or features described hereinabove with reference to FIGS. 2A-D.

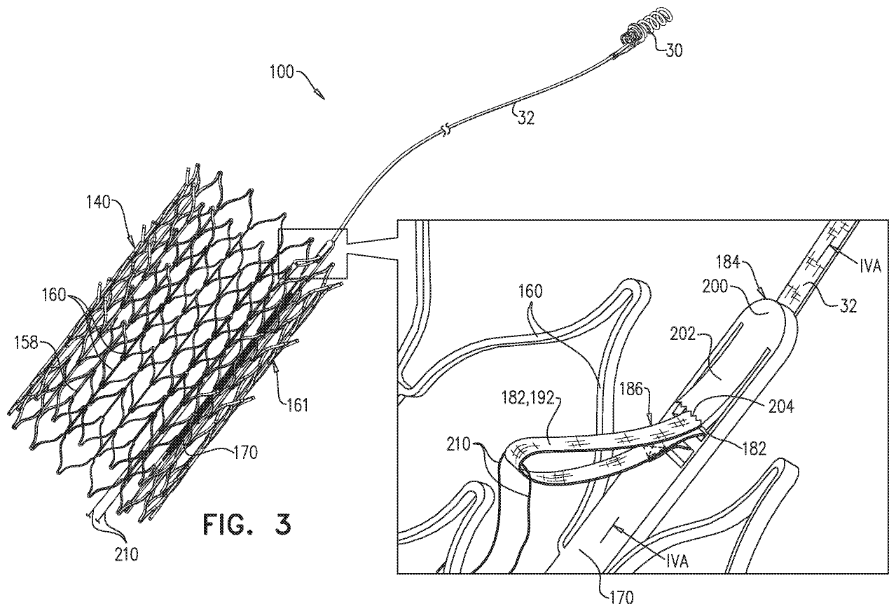

Reference is made to FIG. 3, which is a schematic illustration of a system 100 for treating heart 10, in accordance with an application of the present invention. System 100 comprises first tissue anchor 30, which is configured to be implanted in cardiac tissue of the patient; and a second tissue anchor 140, which is configured to be implanted in the patient, either before or after implanting first tissue anchor 30, such that first and second tissue anchors 30 and 140 are coupled together by one or more tethers 32. First tissue anchor 30 may implement any of the techniques described hereinabove with reference to FIGS. 1A-E and/or 2A-D regarding first tissue anchor 30, mutatis mutandis; second tissue anchor 140 may implement any of the features described hereinabove with reference to FIGS. 1A-E and/or 2A-D regarding second tissue anchor 40, mutatis mutandis; and the one or more tethers 32 may implement any of the features described hereinabove with reference to FIGS. 1A-E and/or 2A-D regarding the one or more tethers 32, mutatis mutandis.

Second tissue anchor 140 comprises a stent 158, which comprises a plurality of struts 160 arranged as a tubular stent body 161. Stent 158 may implement any of the techniques described hereinabove regarding stent 58, mutatis mutandis.

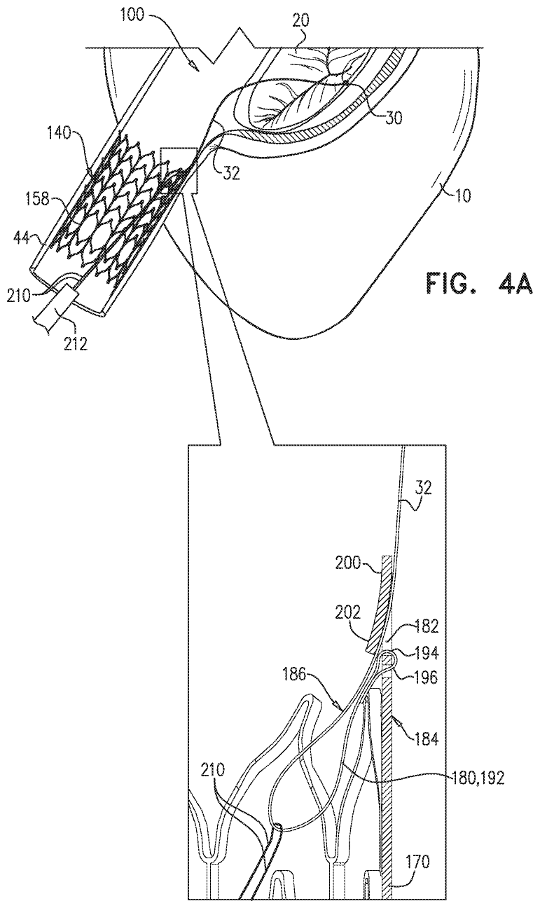

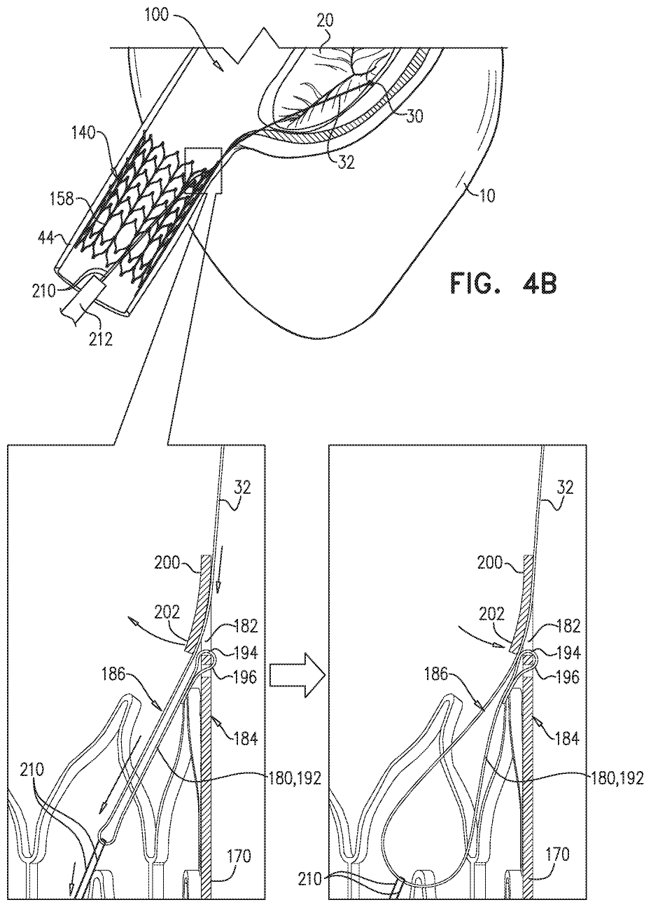

Stent 158 comprises a locking frame 184. For some applications, locking frame 184 is integral with tubular stent body 161, such as shown in FIG. 3 (and FIGS. 4A-B, 5, and 6A-B, described hereinbelow). For example, stent 158 may be manufactured from a single sheet of metal, e.g., using laser cutting or other manufacturing techniques. For other applications, locking frame 184 is fabricated as a separate element that is fixed to tubular stent body 161, such as by welding (configuration not shown). For some applications, at least one of struts 160 is oriented axially (i.e., along the axis of the stent) as a backbone 170 (which may be thicker, wider, and/or stronger than other struts 160 and/or other axially-oriented struts 160). Locking frame 184 is integral with backbone 170, or is fabricated as a separate element that is fixed to backbone 170.

A longitudinal portion 180 of the one or more tethers 32 passes through one or more openings 182 of locking frame 184 so as to form a tether loop 186. System 100 is arranged such that when first tissue anchor 30 is implanted in the cardiac tissue and second tissue anchor 140 is implanted in the patient, enlargement of tether loop 186 by pulling on tether loop 186 applies tension between the first and the second tissue anchors 30 and 140 (using at least a longitudinal portion of the one or more tethers 32).

For some applications, longitudinal portion 180 of the one or more tethers 32 passes through exactly one opening 182 of locking frame 184 so as to form tether loop 186, such as shown in FIG. 3 (and FIGS. 4A-B, 5, and 6A-B, described hereinbelow). (It is noted that another portion of longitudinal portion 180, such as fixation loop 196, described hereinbelow, may additionally pass through one or more openings of locking frame 184; nevertheless, tether loop 186 itself is formed only from longitudinal portion 180 passing through exactly one opening 182.) For other applications, longitudinal portion 180 of the one or more tethers 32 passes through two openings 182 of locking frame 184 so as to form tether loop 186, such as shown in FIGS. 2A-D, mutatis mutandis.

Typically, longitudinal portion 180 of the one or more tethers 32 that forms tether loop 186 is an end longitudinal portion 192 of the one or more tethers 32 (end longitudinal portion 192 extends from an end 194 of the one or more tethers 32 (labeled in FIG. 4A) along a longitudinal portion of the one or more tethers 32, such as for several centimeters or more). For some applications, end longitudinal portion 192 is fixed to locking frame 184, such as shown in FIG. 3 (and FIGS. 4A-B, 5, and 6A-B, described hereinbelow). For example, end longitudinal portion 192 may be arranged so as to define a fixation loop 196, which is fixed to locking frame 184 (fixation loop 196 should not be confused with tether loop 186; the two loops serve unrelated purposes). Alternatively, for some applications, end longitudinal portion 192 is fixed to tubular stent body 161 (configuration not shown).

For some applications, locking frame 184 is shaped so as define a base 200 and a deflectable tab 202, and longitudinal portion 180 of the one or more tethers 32 passes between base 200 and deflectable tab 202. Deflectable tab 202 and locking frame 184 are arranged to allow one-way advancement of the one or more tethers 32 through the one or more openings 182 (e.g., through exactly one opening 182), while inhibiting advancement in the opposite direction. Optionally, deflectable tab 202 is shaped so as to define serrations 204.

Reference is made to FIGS. 4A-B, which are schematic illustrations of a technique for treating heart 10 using system 100, in accordance with an application of the present invention. As shown in FIG. 4A, during a first stage of an implantation procedure, first tissue anchor 30 is implanted in cardiac tissue of the patient, and second tissue anchor 140 is implanted in the patient, either before or after implanting first tissue anchor 30, such that first and second tissue anchors 30 and 140 are coupled together by the one or more tethers 32 and longitudinal portion 180 of the one or more tethers 32 passes through the one or more openings 182 of locking frame 184 so as to form tether loop 186. For example, first tissue anchor 30 may be implanted in the vicinity of tricuspid valve 20 (as shown), e.g., on or near the annulus, and/or second tissue anchor 140 may be implanted in a superior vena cava (SVC) 42, an inferior vena cava (IVC) 44 (as shown), or a coronary sinus 46. Typically, first and second tissue anchors 30 and 140 are implanted in a transcatheter procedure (typically endovascularly, such as percutaneously), via one or more catheters, such as described hereinabove with reference to FIGS. 1A-E and/or in the applications incorporated hereinbelow by reference. Optionally, the one or more tethers 32 comprise two tethers 32 that are coupled together in situ during the first stage of the implantation procedure, such as using techniques described in one or more of the applications incorporated by reference hereinbelow, e.g., using the techniques described with reference to FIGS. 20-26 of U.S. Pat. No. 9,307,980. Typically, first tissue anchor 30 is configured to penetrate the cardiac tissue. For some applications, first tissue anchor 30 comprises a helical tissue-anchoring element, or one of the anchors described in PCT Publications WO 2016/087934 and/or WO 2016/18939, which are incorporated herein by reference.

For some applications, at this first stage of the implantation procedure, the one or more tethers 32 are slack, i.e., do not apply tension between first and second tissue anchors 30 and 140.

As shown in FIG. 4B, thereafter, during a second stage of the implantation procedure, tension is applied between first and second tissue anchors 30 and 140 by enlarging tether loop 186 by pulling on tether loop 186. For example, a flexible elongate member 210 (e.g., a suture, string, or wire) may removably engage tether loop 186, such as by being looped through tether loop 186, and tension may be applied to flexible elongate member 210 by pulling on one or more ends of flexible elongate member 210 outside the body of the patient. Typically, flexible elongate member 210 removably engages tether loop 186 before the tether loop is introduced into the body of the patient.

Alternatively, flexible elongate member 210 is removably engaged with tether loop 186 after the tether loop is introduced into the body of the patient, such as for application in which the tension is applied after allowing at least 24 hours for tissue growth, such as described hereinbelow. After the tension is applied, flexible elongate member 210 is disengaged from tether loop 186, such as by pulling on one end of the flexible elongate member outside the body, and removed from the body. Typically, flexible elongate member 210 passes through a catheter 212. Alternatively, tether loop 186 is pulled using another element of a delivery system, such as a hook and/or rod.

Typically, application of the tension remodels tricuspid valve 20, by drawing two or three of the leaflets together to enhance coaptation.

For some applications, the tension is applied after allowing at least 24 hours (e.g., at least one week, such as at least one month) for tissue growth (e.g., fibrous and/or endothelial tissue growth) on first tissue anchor 30 to strengthen anchoring of first tissue anchor 30 in the cardiac tissue. For some applications, the tension is applied within two months after implanting first tissue anchor 30.

For other applications, the implantation procedure is a single surgical session that includes both the first stage (during which first tissue anchor 30 and second tissue anchor 140 are implanted) and the second stage (during which the tension is applied). For example, the tension may be applied within three hours after implanting the first tissue anchor.

Use of the techniques described herein may allow the use of longer tether(s) 32 than would otherwise be possible. Using longer tether(s) 32 may afford greater flexibility in selecting the axial location for implantation of stent 158 in SVC 42, IVC 44, or coronary sinus 46, because the axial location can be selected without regard to the exact desired tension that will ultimately be applied between the first and the second tissue anchors. In addition, the flexibility of selecting the axial location without regard to the exact desired tension may allow releasing the entire stent from a delivery catheter essentially at once, rather than retaining a portion of the stent radially compressed to allow axial motion of the stent after partial release in order to set the tension. The release of the stent from the delivery catheter essentially at once may allow the use of a shorter stent than would be necessary for staged release from the delivery catheter.

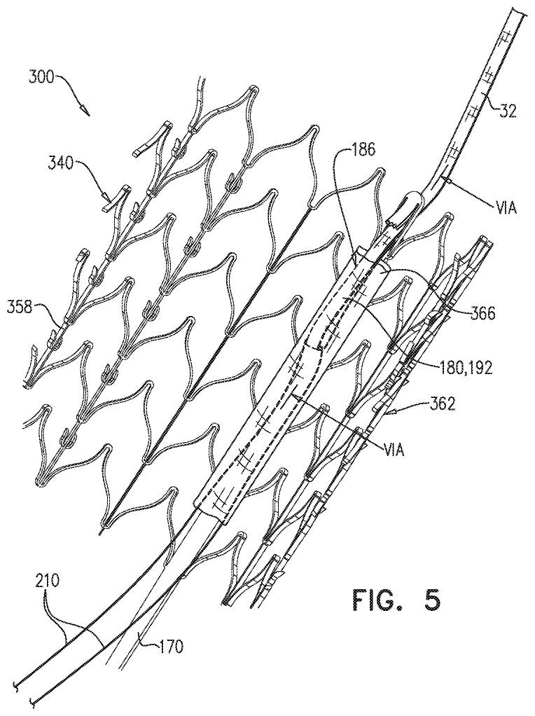

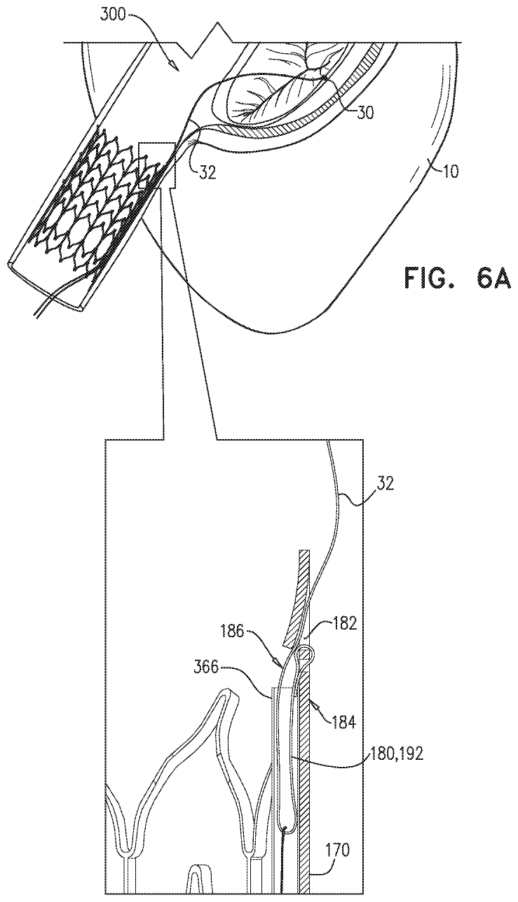

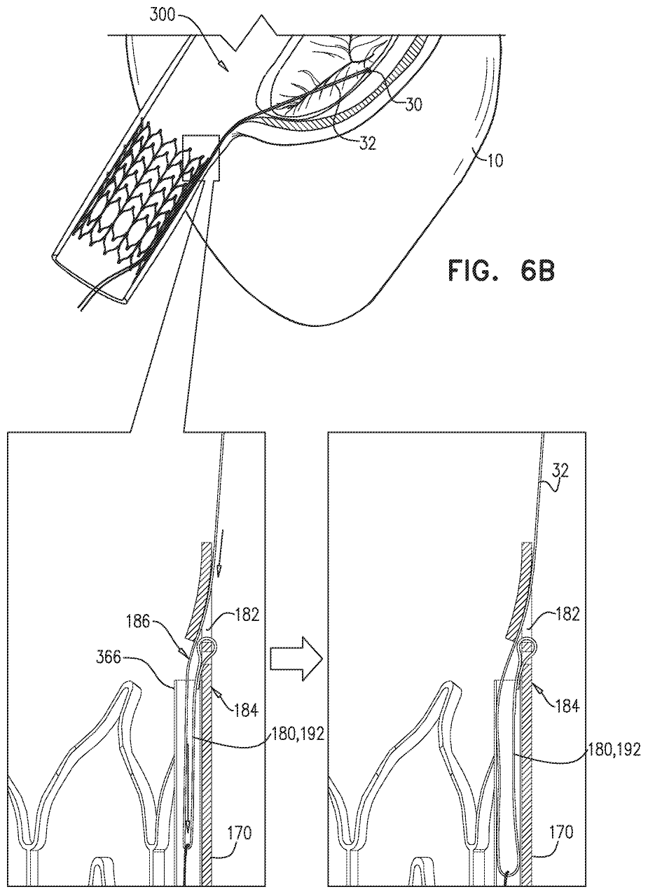

Reference is made to FIG. 5, which is a schematic cross-sectional illustration of a portion of a system 300 for treating heart 10, in accordance with an application of the present invention. Reference is also made to FIGS. 6A-B, which are schematic illustrations of a technique for treating heart 10 using system 300, in accordance with an application of the present invention. Other than as described below, system 300 is identical to system 100, described hereinabove with reference to FIGS. 3-4B, and may implement any of the features thereof.

A stent 358 of a second tissue anchor 340 of system 300 comprises a flop-prevention sleeve 366 disposed inside a tubular stent body 362 of stent 358. Tether loop 186 is disposed partially within flop-prevention sleeve 366. Flop-prevention sleeve 366 holds tether loop 186 from flopping around within tubular stent body 362. For applications in which flexible elongate member 210 is used to pull on tether loop 186, such as described hereinabove with reference to FIG. 4B, flexible elongate member 210 is partially disposed within flop-prevention sleeve 366.

For some applications, fibrous glue is applied to the tissue-coupling elements of the tissue anchors described herein to help secure the anchors in place and minimize detachment. Optionally, tissue-growth-enhancing coating is also applied to the tissue-coupling elements, as described hereinabove.

Reference is made to FIGS. 2A-D, 3, 4A-B, 5, and 6A-B. In an application of the present invention, instead of locking frame 84 or 184, the system comprises a cinching element, such as a cinching bead, and the longitudinal portion of the one or more tethers 32 passes through the cinching element to form the tether loop. This configuration may be implemented, mutatis mutandis, in combination with any of the features described herein, including, but not limited to, flop-prevention sleeve 366, described with reference to FIGS. 5 and 6A-B.

The scope of the present invention includes embodiments described in the following applications, which are assigned to the assignee of the present application and are incorporated herein by reference. In an embodiment, techniques and apparatus described in one or more of the following applications are combined with techniques and apparatus described herein: U.S. Pat. No. 8,475,525 to Maisano et al.; U.S. Pat. No. 8,961,596 to Maisano et al.; U.S. Pat. No. 8,961,594 to Maisano et al.; PCT Publication WO 2011/089601; U.S. Pat. No. 9,241,702 to Maisano et al.; PCT Publication WO 2013/011502; U.S. Provisional Application 61/750,427, filed Jan. 9, 2013; U.S. Provisional Application 61/783,224, filed Mar. 14, 2013; PCT Publication WO 2013/179295; U.S. Provisional Application 61/897,491, filed Oct. 30, 2013; U.S. Provisional Application 61/897,509, filed Oct. 30, 2013; U.S. Pat. No. 9,307,980 to Gilmore et al.; PCT Publication WO 2014/108903; PCT Publication WO 2014/141239; U.S. Provisional Application 62/014,397, filed Jun. 19, 2014; PCT Publication WO 2015/063580; US Patent Application Publication 2015/0119936; U.S. Provisional Application 62/086,269, filed Dec. 2, 2014; U.S. Provisional Application 62/131,636, filed Mar. 11, 2015; U.S. Provisional Application 62/167,660, filed May 28, 2015; PCT Publication WO 2015/193728; PCT Publication WO 2016/087934; US Patent Application Publication 2016/0242762; PCT Publication WO 2016/189391; US Patent Application Publication 2016/0262741; U.S. Provisional Application 62/376,685, filed Aug. 18, 2016; U.S. Provisional Application 62/456,202, filed Feb. 8, 2017; International Application PCT/US2018/017352, filed February 8, 2018; and U.S. application Ser. No. 15/891,664, filed Feb. 8, 2018.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. A system for treating a heart of a patient, comprising:
a first tissue anchor, which is configured to be implanted in cardiac tissue of the patient;
a second tissue anchor, which (a) is configured to be implanted in the patient, and (b) comprises a stent, which comprises a plurality of struts arranged as a tubular stent body and
one or more tethers that couple together the first and the second tissue anchors,
wherein a longitudinal portion of the one or more tethers passes through one or more openings of a locking frame so as to form a tether loop, and
wherein the system is arranged such that when the first tissue anchor is implanted in the cardiac tissue and the second tissue anchor is implanted in the patient, enlargement of the tether loop by pulling on the tether loop applies tension between the first and the second tissue anchors, characterized in that:
the stent comprises the locking frame, the locking frame being shaped so as to define a base and a deflectable tab,
the longitudinal portion of the one or more tethers passes between the base and the deflectable tab, and
the deflectable tab and the locking frame are arranged to allow one-way advancement of the one or more tethers through the one or more openings, while inhibiting advancement in the opposite direction.

2. The system according to claim 1, wherein the locking frame is integral with the tubular stent body.

3. The system according to claim 1, wherein the longitudinal portion of the one or more tethers pass through exactly one opening of the locking frame so as to form the tether loop.

4. The system according to claim 1, wherein the first tissue anchor is configured to penetrate the cardiac tissue.

5. The system according to claim 1, wherein the longitudinal portion of the one or more tethers that forms the tether loop is an end longitudinal portion of the one or more tethers.

6. The system according to claim 5, wherein the end longitudinal portion is fixed to the locking frame.

7. The system according to claim 5, wherein the end longitudinal portion is fixed to the tubular stent body.

8. (canceled)

9. The system according to claim 1, wherein the deflectable tab is shaped so as to define serrations.

10. The system according to 7 1, wherein the stent comprises a flop-prevention sleeve disposed inside the tubular stent body, and wherein the tether loop is disposed partially within the flop-prevention sleeve.
